(19) United States
(12) Patent Application Publication
(10) Pub. No.: US 2017/0158535 A1
(43) Pub. Date: Jun. 8, 2017

(54) PROCESS FOR RECOVERING ELEMENTAL SELENIUM FROM WASTEWATER

(71) Applicant: Wageningen Universiteit, Wageningen (NL)

(72) Inventors: Simon Petrus Wilhelmus HAGEMAN, Arnhem (NL); Renata Dorothea VAN DER WEIJDEN, Arnhem (NL); Cees Jan Nico BUISMAN, Harich (NL)

(73) Assignee: Wageningen Universiteit, Wageningen (NL)

(21) Appl. No.: 15/325,642

(22) PCT Filed: Jul. 6, 2015
(86) PCT No.: PCT/NL2015/050492

§ 371 Date: Jan. 11, 2017

(30) Foreign Application Priority Data
Jul. 14, 2014 (EP) 14176948.9

(51) Int. Cl. C02F 3/34; C01B 19/02; C12P 3/00; C02F 3/28; C02F 1/70

(52) U.S. Cl. CPC C02F 3/345; C02F 3/28; C02F 1/70; C12P 3/00; C01B 19/02; C02F 2101/106

(57) ABSTRACT

A first aspect of the present invention relates to a process for recovering crystalline elemental selenium (Se) from an aqueous composition, such as waste water or groundwater. A second aspect of the present invention further relates to a microbial sludge comprising crystalline elemental selenium, which sludge may be used in the further recovery of elemental selenium.

### TECHNICAL FIELD OF THE INVENTION

The present invention relates to a process for recovering elemental selenium (Se) from an aqueous composition, such as wastewater or groundwater. The present invention further relates to a microbial sludge comprising elemental selenium, which sludge may be used in the further recovery of elemental selenium.

### BACKGROUND OF THE INVENTION

Selenium is a naturally occurring metalloid element having atomic number 34 and an atomic weight of 78.96. It lies between sulfur and tellurium in group 16 and between arsenic and bromine in period 4 of the periodic table of elements. Selenium is widely dispersed in igneous rock. In hydrothermal deposits, it is associated isothermally with silver, gold, antimony, and mercury. Selenium also appears in large quantities, but in low concentrations, in sulfide and porphyry copper deposits. Moreover, selenium is widely associated with various types of sedimentary rock, but also in oil and coal.

Selenium forms covalent compounds with many other substances and is necessary in small amounts for most forms of life. However, selenium is a chemical analog of sulfur and can interfere with normal cellular metabolism. Selenium accumulates in the bodies of plants and fish that live in selenium contaminated water and in the bodies of wildlife and humans that eat those plants and fish. In humans, elevated selenium concentrations may cause neurological damage and hair and nail loss. In this regard, the water soluble forms of selenium selenate (SeO₄²⁻) and selenite (SeO₃²⁻) are particularly harmful and should thus be removed from wastewater as much as possible (Lemly, A. D., in Ecotoxicology and Environmental Safety 2004, 59, (1), pp.

44-56).

However, removal of selenate and selenite from large volumes of water is one of the more complex environmental problems. Several methods have been investigated for treating agricultural wastewaters. These treatments generally involve the direct reduction of selenate (SeO₄²⁻) to elemental selenium using either chemical reduction with ferrous hydroxide (Fe(OH)₂) or biological reduction using bacteria. The chemical reduction method requires operating conditions that can be difficult to achieve in large treatment operations such as a pH of about 9 and a large excess of Fe(OH)₂.

Other treatment processes, such as lime precipitation, chemical reduction, activated alumina adsorption, ion exchange, reverse osmosis, electrodialysis, or distillation have been demonstrated to remove a variety of pollutants including selenium from water to below drinking water standards. However, these methods are generally expensive and cost prohibitive for the treatment of large volumes of water and produce non-stable products that can leach out of storage facilities.

Moreover, lime precipitation and chemical reduction processes can result in a mixed metal waste product that can increase treatment and disposal costs, and generally require some pH adjustment of the influent. Additionally, laboratory tests and pilot plant studies have shown that chemical precipitation employing alum, lime, ferrous sulfate or ferric sulfate are substantially ineffective for removing selenium in the selenate and selenite oxidation state.

A process for the removal of selenium from waters using a reactor containing microbial sludge has been investigated. However, this method is inadequate because it is intended to treat water that contains at least 0.5 mg/L of oxidized forms of selenium (such as selenate and selenite) and is only capable of lowering the selenium concentration to 10% of the original value. In order to achieve the drinking water standard, a polishing step such as ion exchange is needed. Furthermore, the selenium particles formed are amorphous and very small and therefore difficult to recover and reuse. Moreover, selenium containing waters greatly differ from each other in type of selenium contamination, levels of selenium, types of co-contaminants, levels of co-contaminants, pH, ionic content, etc. These factors make the bioremediation of selenium containing waters very complex and low yielding.

Hence, a need remains for a process which is able to cost-efficiently remove selenium or its soluble forms selenate and/or selenite, without having to use environmentally hazardous materials and produce reusable products.

### SUMMARY OF THE INVENTION

The present invention relates to a process for recovering elemental selenium (Se) from an aqueous composition, the process comprising the steps of:
a. providing an aqueous composition comprising selenite;
b. reacting at least a part of the selenite with sulfide into selenium sulfide;
c. reducing with a microbial sludge at least a part of the selenium sulfide formed into hydrogen sulfide and crystalline elemental selenium;
d. separating at least a part of the microbial sludge comprising the crystalline elemental selenium formed from the aqueous composition.

With the process of the present invention it has now for the first time been made possible to efficiently recover, by means of a biological treatment, elemental selenium from selenium polluted aqueous compositions, such as wastewater. Furthermore, the produced selenium particles are extracellular, more pure, crystalline and hexagonal and larger in size compared to amorphous selenium. This allows far better selenium separation opportunities from the sludge when compared to traditional sludges comprising amorphous selenium.

In this process toxic selenite is first precipitated with sulfide to form the insoluble product selenium sulfide (SeS₂). SeS₂ consists of elemental selenium and elemental sulphur. The average S/Se ration in SeS₂ depends on reaction conditions, however in general the ratio S/Se varies between 1.7 and 2.2 (Geoffroy et al., Journal of harzardous materials 2011, 185, (1), 148-154.).

After the water insoluble selenium sulfide has been formed and has precipitated from the aqueous composition, the precipitated selenium sulfide is reduced by micro-organisms present in the provided microbial sludge to sulfide and crystalline elemental selenium. During this process the micro-organisms grow and form more microbial sludge, moreover most of the crystalline elemental selenium formed is found in said sludge.

In a subsequent step at least a part, but preferably all of the microbial sludge comprising the crystalline elemental selenium is separated from the aqueous composition. The sludge may be further processed, e.g. by heating, melting or gravitational separation to obtain crystalline elemental selenium.

Optionally, prior to step a) at least a part of any selenate present in a polluted aqueous composition is converted into selenite. As mentioned above, both selenate and selenite are commonly found in wastewater and are very soluble in water. However, since selenate does not readily react with sulfur to selenium sulfide, it should first be converted into selenite. Processes for reducing selenate to selenite are well known in the art (Hageman et al, Water Research, 47 (2013) 2118-2128; Oremland et al, Applied and Environmental Microbiology 60 (1994), 3011-3019 Chemically:Bye and Lund, Journal of analytical Chemistry 332 (1988), 332, 242-244) and may thus be readily employed to prepare an aqueous composition comprising selenite, i.e. a composition as referred to in step a) of the present process.

A second aspect of the present invention relates to a microbial sludge comprising crystalline elemental selenium obtainable by the process of the present invention. Unlike known sludges, the sludge according to the present invention comprises relatively high amounts of crystalline elemental selenium. Moreover, the produced selenium particles are extracellular, more pure, hexagonal (i.e. crystalline) and larger in size compared to amorphous selenium. This allows far better selenium separation opportunities from the sludge when compared to traditional sludges comprising amorphous selenium.

The sludge obtained with the process of the present invention may be processed further to recover the elemental selenium present in said sludge.

### Definitions

The term “aqueous composition” as used herein has its conventional meaning and refers to a composition which comprises at least 80% by weight water, preferably at least 90% by weight water. Within the context of the present invention the term aqueous composition also encompasses wastewater, groundwater or surface water.

The term “wastewater” as used herein has its conventional meaning and refers to water that has been adversely affected in quality by anthropogenic influence or by means of natural pollution, e.g. from sediments which are washed.

The term “reducing” as used herein as its conventional meaning and refers to a chemical reaction wherein a molecule which is reduced receives electrons and experiences a decrease in its oxidation state. Within the context of the present invention the reaction of selenium sulfide into selenium and hydrogen sulfide is considered a reduction reaction.

The term “microbially reducing” or “reducing with a microbial sludge” as used herein refers to the chemical reduction of a compound, such as selenium sulfide, as facilitated by micro-organisms.

The term “microbial sludge” or “biosolids” as used herein has its conventional meaning and refers to protoplasm produced by micro-organisms (Davis M. L., Water and Wastewater Engineering, 2010).

The term “volume/surface average particle diameter” as used herein has its conventional meaning and refers to the so called Sauter mean diameter (d32) determinable with light scattering.

### DETAILED DESCRIPTION OF THE INVENTION

A first aspect of the present invention relates to a process for recovering elemental selenium (Se) from an aqueous composition, the process comprising the steps of:
a. providing an aqueous composition comprising selenite;
b. reacting at least a part of the selenite with sulfide or a source thereof into selenium sulfide;
c. reducing with a microbial sludge at least a part of the selenium sulphide formed into hydrogen sulfide and crystalline elemental selenium;
d. separating at least a part of the microbial sludge comprising the crystalline elemental selenium formed from the aqueous composition.

With the process of the present invention it has now for the first time been made possible to separate from an aqueous composition selenite (and optionally selenate) and to recover elemental selenium from said composition. Furthermore, the produced selenium particles are extracellular, pure, hexagonal (i.e. crystalline) and larger in size compared to amorphous selenium. This allows far better selenium separation opportunities from the sludge when compared to traditional sludges comprising amorphous selenium.

In this process the toxic selenite is first precipitated with sulphide to form the insoluble product selenium sulfide (SeS₂). The average S/Se ration in SeS₂ depends on reaction conditions, however in general the ratio S/Se varies between 1.7 and 2.2 (Geoffroy et al., Journal of harzardous materials 2011, 185, (1), 148-154.).

Preferably, as a sulfide source a sulfide salt or a hydrate thereof is used, most preferably sodium sulfide (Na₂S) or its hydrate is used.

In a further step, the selenium sulfide is reduced (e.g. in a bioreactor) by micro-organisms present in the sludge to sulfide and elemental selenium. During this process the micro-organisms grow and form more microbial sludge. Most of the crystalline elemental selenium formed is found in said sludge

As a starter-culture for this reaction an anaerobic microbial sludge may be used, which comprises sulfate reducing micro-organisms. The microbial sludge used preferably comprises complete oxidizing sulphate reducers. Preferred oxidizing sulphate reduces used are from the genus Desulfobacter gen. nov. Desulfobacter postgatei sp. nov, (Friedrich Widdel and Norbert Pfennig , 1981 Arch Microbiol (1981) 129:395-400), in particular Desulfobacter postgatei, Desulfotomaculum acetoxidans Desulfobacterium, Desulfotomaculum and Desulfococcus species and Desulfobacca acetoxidans. Microorganisms. The reaction is preferably carried out at a pH in the range between pH 4 to 10 and at a temperature between 10° C. to 75° C.

A highly preferred microbial sludge which may be used as a starter-culture for the microbial reduction of selenium sulfide is the so-called Emmtec sludge, the features of this sludge have been described in amongst others d'Abzac, P et al., Environmental science & technology 2009, 44, (1), 412-418.

The sludge used as a starter culture is preferably formulated in granulates. This makes the sludge more easy to handle and facilitates dosing.

The aqueous composition as referred to in step a) of the present invention is preferably wastewater. Particularly in wastewater from coal fired power plants, oil refineries, metal operations (copper, zinc, nickel, gold), acid mine drainage and in agricultural drainage water and groundwater the levels of selenite and/ selenate can be relatively high. Typically, the concentrations selenite and/or selenate range between 0.1 and 4000 mg/L. Since both selenite and selenate can be toxic it is important to remove at least a part of these compounds from the wastewater. The aqueous composition may be untreated wastewater, however it is also possible to use the effluent of wastewater which has already been subjected to one or more treatment steps, such as a presedimentation stage. In this regard reference is made to the PhD thesis of Lenz, M., Biological selenium removal from wastewaters, 2008.

One possible treatment step which is preferably carried out prior to step a) of the process of the present invention is the conversion of any selenate present in the aqueous composition (e.g. wastewater) into selenite. As mentioned above, both selenate and selenite are commonly found in wastewater and are very soluble in water. However, since selenate does not readily react with sulfide to selenium sulfide, it should first be converted into selenite.

Processes for reducing selenate to selenite are well known in the art and may thus be readily employed to prepare an aqueous composition comprising selenite, i.e. a composition as referred to in step a) of the present process. Most preferably, such a reduction step of selenate into selenite is carried out by micro-organisms in a bio-reactor. In such as case the process would comprise the following steps:
a. providing an aqueous composition comprising oxidized forms of selenium, such as selenate and selenite;
b. reducing with a microbial sludge at least a part of the oxidized forms of selenium, in particular selenate, into selenite
c. reacting at least a part of the selenite with sulfide or a source thereof into selenium sulfide;
d. reducing with a microbial sludge at least a part of the selenium sulfide formed into hydrogen sulfide and crystalline elemental selenium;
e. separating at least a part of the microbial sludge comprising the crystalline elemental selenium formed from the aqueous composition.

With the process of the present invention it has now also become possible to substantially remove oxidized forms of selenium (such as selenate and selenite)from aqueous compositions comprising less than 0.100 mg/L, preferably less than 0.005 mg/L of such soluble forms of selenium. Hence, it is now possible to also economically recover selenium form less polluted wastewater. This is a major advantage, because now it is has become possible to treat wastewater which has relatively low levels of selenate and selenite, but which nevertheless poses a health risk due to the accumulation of these compounds in humans.

A second aspect of the present invention relates to a microbial sludge obtainable by the above mentioned method. Other then known microbial sludges, this sludge comprises a relatively high amount of crystalline elemental selenium up to 90%. Production of (XRD) hexagonal selenium crystals, extracellular selenium particles and particles that are separable from the water phase through gravitational sedimentation. Remarkably, the particles have a volume/surface average particle diameter (d32) in the range of 0.1 to 100 μm, preferably 1-50 μm, most preferably 1-10 μm. This makes them particularly easy to separate from the sludge.

The present invention will now be illustrated further by means of the following non-limiting examples.

### EXAMPLES

### Material & Methods

### Biomass

The inoculums (i.e. starter culture) was granular Emmtec sludge. Emmtec sludge originates from a reactor in which sulphate is reduced to sulphide with ethanol as electron source. EmmTec sludge contains 19.9 (+/−12.3) mg sulphur in the total suspended solid 16.

### Dry Weight Determination

Wet weight biomass was washed with demi water. Dry weight was determined in triplicate by drying 5 g washed wet biomass at 105° C. for 3 days.

### Medium

Crimp-seal flasks with an average internal volume of 118 mL were filled with 50 mL medium. The medium was based as described in Stams et al., in Applied and Environmental Microbiology 1992, 58, 1, pp 346-352) and consisted of (in grams per litre): Na₂HPO₄.2H₂O (0.53), KH₂PO₄ (0.41), NH₄Cl (0.3), CaCl₂.2H₂O (0.11), MgCl₂. 6H₂O (0.10), and acid- and base trace elements and vitamin solution. Sodium selenite as a trace element, sodium hydrogen carbonate and sodium sulphide were not added. The medium pH was set to 7 with NaOH. A volume of 5 mL 100 mM sodium lactate was added as electron donor.

### Example 1: Reduction of Commercial SeS₂

The electron acceptor was commercial crystalline SeS₂ from Sigma Aldrich. Experiments with biomass, electron donor and SeS₂ were performed in batch bottles. An overview is given in table 2 and listed in the first sequence (Bottles I-a to I-d). The following control experiments were added; #I-a, without any biomass; #I-d with Emmtec sludge and without Se5₂. The total volume is calculated with the assumption that the wet weight sludge density was 1 g/L and the SeS₂ powder volume in the water phase was negligible. The headspace was degassed to 0.5 atm and subsequently raised by increasing the pressure in the bottle to a overpressure of 0.5 atm. with N₂. This gas pressure cycle was repeated 5 times and ended with a overpressure of 0.5 atm. N₂. The bottles were placed in an orbital shaker (100 rpm) at 30° C. for 36 days.

A total liquid volume of ±7 mL was taken for sampling by a needle and syringe during the experiment. Liquid samples were centrifuged 10.000 RPM for 10 minutes table centrifuge, (Microlite Thermo ICE) and the supernatant was analysed on total sulphide concentration in the liquid (S²⁻_{T}(aq)) (=H₂S(aq)+HS⁻(aq)+S²⁻(aq)) and the pellets of bottle #I-b and #I-c were analysed on ratio S/Se by ICP-OES.

At the end of the experiment the headspace pressure was measured and analysed by GC for fraction carbon dioxide and fraction methane at room temperature. The liquid volume was measured by the difference in weight by the full bottle and the empty bottle. An 4 mL liquid sample was taken to measure the final S²⁻_{T}(aq) and the chemical oxygen demand (COD), the remainder was stored in the freeze. In total, around 7+4 mL was used for sampling and this corresponds with 20% starting volume. The precipitate colour was noted.

After opening the bottle in the fume hood, the pH was measured immediately. The reminder batch liquid was stored at 4° C. for one night in a closed 50 mL tube. The next morning, the granular biomass particles used as inoculum (>1 mM) were separated with a sieve (1 mm). The granules were rinsed with aqua to remove any attached amounts of S or Se. The filtrate and the water from rinsing the granules were raised to a total volume of 50 mL and contained most of the selenium, sulphur and disperse biomass. This 50 mL was centrifuged at 2395 RPF 3660 RPM at 20° C. for 10 minutes and the pellet was mixed in 20 mL milliQ. This mixture was analysed with macro- and micro-scope. Pictures of the precipitate were taken with a Nikon SMZ 800 macroscope and a Nikon Eclipse E400 microscope. Next, the sample used for macro-scope and the reminder of the 20 mL liquid were centrifuged at 2395 RPF 3660 RPM at 20° C. for 10 minutes and the pellet was air dried at room temperature for three days. Hot tap water (estimated to be 60° C.) was used to dry the samples of #1-6 and all from series #2 au bain marie. A fraction of the dried pellet was used to determine the sulphur and selenium content with ICP-OES analyses.

Prior to XRD analyses, also a fraction of the dried pellet was re-suspended in MilliQ and the suspension was transported on an object glass (used for microscopy slide VWR ECN 631-1550 76×26×1 mm) and dried at room temperature.

### Example 2a: Synthesis of Selenium Sulphide Suspension

Effluent from a bioreactor converting selenate to selenite at pH=6 and T=30° C. was stored at −20° C. More details about the reactor performance are described in Hageman et al in Water Reseach, 2013. After unfreezing the samples, solid particles in the effluent were first removed by centrifugation at 45000 RPM for 20 min with Firlabo SW12 centrifuge. The supernatant was filtered (Schleicher & Schuell Ref. no. 300012) and then analysed for total selenium (ICP-OES) and selenite (IC) in duplicate. A volume of 226 mL of this filtered supernatant was mixed in seconds with 1.22 g Na₂S 9H₂O dissolved in 10 mL milliQ water. The glass beaker with sulfide was rinsed with 5 mL milliQ water and this volume was also added to the reaction mixture. The reaction solution in the Erlenmeyer was shaken manually. The pH raised to 12 and was adjusted by adding 3 mL 25% HCl, but an overshoot was created (pH=3.53) and this was compensated with the addition of 0.6 mL 0.1 M NaOH to get finally a pH of 5.9 at t=23 minutes. The solution was stored aerobically overnight in a fume hood to remove unreacted sulfide. The next morning a sample of 15 mL was centrifuged at 45000 RPM 20 min with Firlabo SW12 centrifuge. The reaction solution and the supernatant were investigated on selenium content by duplicate ICP-OES measurments. The supernatant was also analysed on sulfide (duplicate) and selenite. The pellet was washed twice with milliQ water prior to selenium and sulfur analyses. Furthermore, during washing, it was noticed that the pellet was fragile and difficult to separate from the supernatant after centrifugation without partly remixing the pellet. However, a part of the pellet sticked to the bottom of the centrifuge tube and was difficult to resuspend in water. Therefore, the produced SeS₂ suspension was directly used in the reduction experiments to avoid segregation of the precipitated SeS₂. The precipitation reaction occurred in old medium (used in the selenate to selenite converting bioreactor) and this reaction solution is added to the batch bottles.

### Example 2b Reduction of Synthesized SeS₂

Synthesized SeS₂ was prepared as described in Example 2a and 50 mL of this SeS₂ suspension in used medium was transferred to each crimp-seal flasks. A volume of 5 mL 100 mM sodium lactate was added and Emmtec sludge was used (sequence two in table 1) since this biomass had a good SeS₂ reducing capacity with commercial SeS₂. The experiments were performed as described in the experimental procedure for commercial SeS₂ reduction. An overview of the synthesized SeS₂ reduction is listed in table 1 (bottle #11-a to #II-d).

### Results

The results of Examples 1 and 2a and 2b are depicted in tables 1 and 2 below.

TABLE 1
composition of the test-bottles prior to microbial reduction of SeS₂

| | | | | | | | | |
|---|---|---|---|---|---|---|---|---|
| | | | | | | synthesized | | |
| | | | | | | Se—S | | |
| bottle | | | Fresh | | Commercial | (mL | Se—S | |
| Nr | | Lactate | Medium | Emmtec | Se—S | old | (mM | Tot V |
| # | Label | (mL) | (mL) | (g ww) | (mg) | medium) | Se) | (mL) |

Experiment sequence # 1 (8 bottles) commercial SeS₂

| | | | | | | | |
|---|---|---|---|---|---|---|---|
| I-a | Control | 5 | 50 | | 71.5 | 9.1 | 55.0 |
| | Commercial SeS₂ | | | | | | |
| I-b | Emmtec | 5 | 50 | 0.5 | 70.6 | 8.9 | 55.5 |
| | Commercial SeS₂ | | | | | | |
| I-c | Emmtec | 5 | 50 | 0.5 | 73.0 | 9.2 | 55.5 |
| | Commercial SeS₂ | | | | | | |
| | duplicate | | | | | | |
| I-d | Control | 5 | 50 | 0.5 | | 0.0 | 55.5 |
| | Emmtec | | | | | | |

Experiment sequence #2 (4 bottles) synthesized SeS₂

| | | | | | | | |
|---|---|---|---|---|---|---|---|
| II-a | Emmtec | 5 | | 0.5 | 50 | 9.5¹ | 55.5 |
| | prepared SeS₂ | | | | | | |
| II-b | Emmtec | 5 | | 0.5 | 50 | 9.5¹ | 55.5 |
| | prepared SeS₂ | | | | | | |
| | duplicate (II-a) | | | | | | |
| II-c | Control | 5 | | | 50 | 9.6¹ | 55.0 |
| | Prepared SeS₂ | | | | | | |
| II-d | Emmtec | 5 | 50 | 0.5 | | 0.0¹ | 55.5 |
| | Control | | | | | | |

TABLE 2
SeS₂ conversion and by-products after microbial reduction

| | SeS₂ | | | | | | | | | | | |
|---|---|---|---|---|---|---|---|---|---|---|---|---|
| | added at | | | S | COD | | % Se in | | | | | |
| Bottle | t = 0 | | Se recovery | recovery | Recov- | | pellet | | ΣCO₂ | | | Ratio |
| Label | (mmole | S²⁻_{T} | (max ≈ 80%)¹⁾²⁾ | (max ≈ 80%)¹⁾²⁾ | ery¹⁾ | Colour | (% | CH_{4,gas} | (mmole) | pH | Pressure | S/Se |
| (—) | Se) | (mmole) | (%) | (%) | (%) | (—) | W/W) | (mmole) | (mmole) | (—) | (mbar) | (—) |
| I-a | 0.50 | 0.10 | 70 | 81 | 85 | orange | 51 | 0.00 | 0.24 | 6.76 | 1486 | 1.89 |
| I-b | 0.49 | 0.52 | ³⁾ | ³⁾ | 73 | black | 75 | 0.03 | 0.48 | 6.49 | 1576 | 0.24 |
| I-c | 0.51 | 0.61 | 79 | 74 | 69 | black | 85 | 0.02 | 0.50 | 6.44 | 1584 | 0.35 |
| I-d | 0.00 | 0.00 | — | — | 88 | — | — | 0.29 | 0.38 | 7.1 | 1491 | — |
| II-a | 0.53 | 0.40 | 86 | 52 | 45 | black/brown | 77 | 0.00 | 0.55 | 6.27 | 1741 | 0.31 |
| II-b | 0.53 | 0.35 | 78 | 39 | 80 | black/brown | 80 | 0.00 | 0.58 | 6.29 | 1551 | 0.16 |
| II-c | 0.53 | 0.08 | 76 | 61 | 74 | red/brown/ | 64 | 0.00 | 0.27 | 6.23 | 1347 | 1.42 |
| | | | | | | black | | | | | | |
| II-d | 0.00 | 0.00 | — | — | 81 | — | — | 0.56 | 0.57 | 6.76 | 1391 | — |

### Analysis

The supernatant was used for S²⁻_{T}(aq) in (mg/L) analyses with a S²⁻ kit, (Dr. Lange LCK-653). The COD was measured by Hach Lange kit (LCK 314, LCK 414 and LCK 514 with ranges of 15-150; 5-60 and 100-2000 mg O₂/L, respectively).Results were obtained with a Hach Lange Xion 500 spectrophotometer.

Prior ICP-OES measurements, pellets or liquids were transferred to 10 mL aqua regia,. This mixture was treated by micro wave (ETHOS 1; Advanced Microwave Digestion system, milestone) assisted heating (In 4 minutes to 90° C.; in 5 minutes from 90° to 130° C.; in 4 minutes from 130° to 160° C. and finally 15 minutes at 160° C.) in closed Teflon tubes. Subsequently, the total volume was set to 100 mL with milliQ and resulted in 10% aqua regia. Samples were analysed by VARIAN VISTA-NIPX CCD—Simultaneous ICP, sea spray concentric glass nebulizer en cyclonic spray chamber.

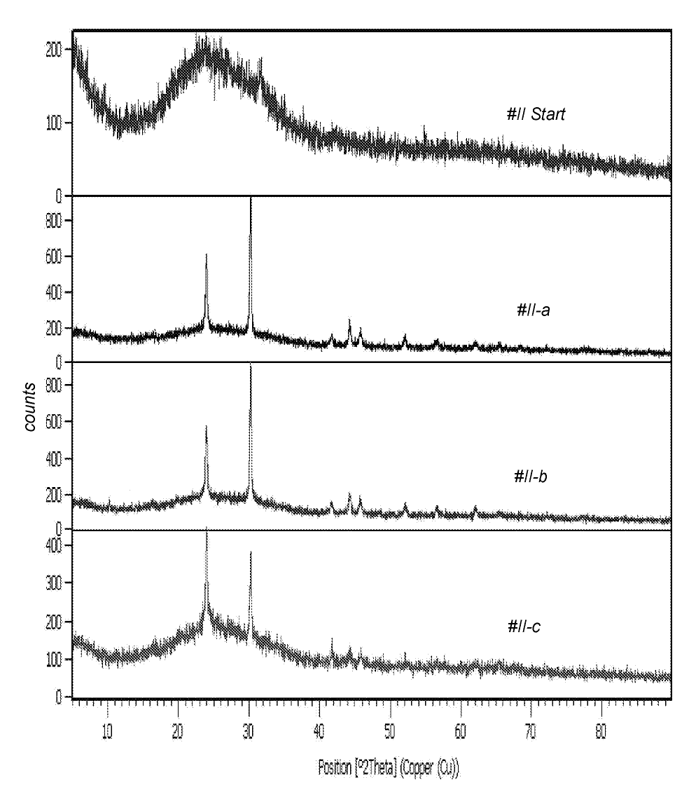

XRD was performed with a PHILIPS SR 4160, 40 kV 30 mA. Raw data was analysed with the program HighScore plus. Peaks were searched with the following method: 2nd derivate; Minimum significance 1.00; Minimum tip width (°2Θ) 0.01; Max. tip width (°2Θ) 1.00 and peak base width (°2Θ) 2.00. The XRD-pattern (FIG. 1; II-start is XRD pattern of the synthesized SeS₂ at prior to its microbial reduction).) were compared with known hexagonal selenium patterns: Peaks were analysed in Search and Match with the following settings: No restrictions, data source: peak data, multi-phase, demote unmatched strong and allow pattern shift.

The IC system consisted of an Autosampler AS-50, a Dionex ICS-2100 equipped with an IonPac AS19 column (4 mm 250 mm) at 30° C., an ASRS 4 mm suppressor (75 mA) and an electrochemical conductivity detector at 35° C. The flow rate was 1.0 mL min and sample loop was 10 μl, but was used in partial loop mode, injecting 5 μl.

The GC system consisted of a Shimadzu 2010 GC equipped with a Porabond Q (50 m×0.53 mm; 10 μm; Varian; Part. no. CP7355) Molsieve 5A (25 m×0.53 mm; 50 μm; Varian; Part. no. CP7538) equipped with a thermal conductivity detector (TCD). Helium was the carrier gas (Carrier gas pressure—lbar) and loop injections of 1.5 mL were used. GC oven temp 75° C., Inlet temp 120° C.; Detector temp 150° C.

### Conclusion

In the process described, selenate is first reduced by microorganisms to selenite and subsequently the toxic selenite is precipitated with sulfide to form the insoluble product SeS₂. Then, the sulphur compound of SeS₂ is reduced by microorganisms to sulfide, while selenium is recovered as crystalline elemental selenium.

Emmtec sludge, originally reducing sulfate to sulfide, was tested for its efficiency to reduce the sulfur compound from a commercially purchased SeS₂. With Emmtec sludge synthesized SeS₂ was also tested: Laboratory SeS₂ was formed with sulfide and selenite from a bioreactor converting selenate to selenite. Removal of the sulfur by reducing sulfur to sulfide with Emmtec sludge was successful for the purchased and the laboratory synthesized SeS₂. The remaining solid consisted of crystalline selenium as determined by XRD. Assumed is that the selenium particle formation was extracellular and the crystalline structure indicated the absence of biomolecules that stabilizes amorphous selenium bio-particles at comparable process conditions in other studies, T=30° C. and pH 6 or 7. The selenium particles seem to be unattached from the biomass.

The obtained results allows the design of a biological process to remove selenate and selenite and to bio-recover selenium since the produced selenium particles are extracellular, pure and hexagonal (i.e crystalline) and this allows better selenium separation opportunities.

1-14. (canceled)

15. A process for recovering elemental selenium (Se) from an aqueous composition, comprising:
(a) providing an aqueous composition comprising selenite;
(b) reacting at least a part of the selenite with sulfide into selenium sulfide;
(c) reducing with a microbial sludge at least a part of the selenium sulfide formed into hydrogen sulfide and crystalline elemental selenium; and
(d) separating least a part of the microbial sludge comprising the crystalline elemental selenium formed from the aqueous composition.

16. The process according to claim 15, wherein the aqueous composition is wastewater or an effluent from a wastewater treatment installation.

17. The process according to claim 15, wherein the aqueous composition comprises between 0.1 and 4000 mg per liter selenite.

18. The process according to claim 15, wherein prior to step (a) at least a part of any selenate present in the aqueous composition is converted into selenite.

19. The process according to claim 18, wherein selenate is converted into selenite by means of a microbial reduction.

20. The process according to claim 19, wherein the microbial reduction is by means of complete oxidizing sulphate reducing micro-organisms.

21. The process according to claim 20, wherein in step (b) the sulfide is a sulfide salt or hydrate thereof.

22. The process according to claim 21, wherein the sulfide salt is sodium sulfide (Na₂S) or a hydrate thereof.

23. The process according to claim 22, wherein in step (c) an anaerobic microbial sludge comprising sulfate reducing micro-organisms is used as a starter-culture.

24. The process according to claim 23, wherein the sulfate reducing micro-organisms comprise complete oxidizing sulfate reducing micro-organisms.

25. The process according to claim 24, wherein the complete oxidizing sulphate reducing micro-organisms are selected from the genus Desulfobacte.

26. The process according to claim 25, wherein the Desulfobacte comprise Desulfobacter postgatei, Desulfotomaculum acetoxidans Desulfobacterium, Desulfotomaculum, Desulfococcus, Desulfobacca acetoxidans or combinations thereof.

27. The process according to claim 15, wherein step (c) is carried out at a pH between 4 to 10 and at a temperature between 10° C. to 75° C.

28. The process according to claim 15, wherein in step (d) the microbial sludge is separated from the aqueous solution by allowing the sludge to settle in a clarifier, such that the crystalline elemental selenium comprising sludge is separated from the aqueous solution.

29. The process according to claim 15, further comprising recovering the crystalline elemental selenium from the microbial sludge.

30. The process according to claim 15, wherein at least a part of the hydrogen sulfide produced in step (c) is reused in step (b).

31. A microbial sludge comprising crystalline elemental selenium obtainable by the process according to claim 15.

32. The microbial sludge according to claim 31, wherein the sludge comprises crystalline particles of elemental selenium having a volume/surface average particle diameter (d32) in the range of 0.1 to 100 μm.

33. The microbial sludge according to claim 32, wherein the sludge comprises crystalline particles of elemental selenium having a volume/surface average particle diameter (d32) in the range of 1-50 μm.

34. The microbial sludge according to claim 33, wherein the sludge comprises crystalline particles of elemental selenium having a volume/surface average particle diameter (d32) in the range of 1-10 μm.
